SEQUENCE LISTING (30 sequences)

SEQ ID NO: 1 - DNA, 18 bp, Artificial Sequence
  Primer
gcctttgctcagtatctt

SEQ ID NO: 2 - DNA, 18 bp, Artificial Sequence
  Primer
aggtttgggttgtcatct

SEQ ID NO: 3 - DNA, 19 bp, Artificial Sequence
  Primer
tatgatgaagcgtttaggc

SEQ ID NO: 4 - DNA, 20 bp, Artificial Sequence
  Primer
cagtaatggacagtttgggt

SEQ ID NO: 5 - DNA, 19 bp, Artificial Sequence
  Primer
cctacggcgtcttctcgac

SEQ ID NO: 6 - DNA, 20 bp, Artificial Sequence
  Primer
ctgcaagaacactctcgcct

SEQ ID NO: 7 - DNA, 21 bp, Artificial Sequence
  Primer
ctggagtcctgtcaggcattg

SEQ ID NO: 8 - DNA, 22 bp, Artificial Sequence
  Primer
cagtcccttgagcagcagataa

SEQ ID NO: 9 - DNA, 18 bp, Artificial Sequence
  Primer
cagcacttcctgaatgag

SEQ ID NO: 10 - DNA, 23 bp, Artificial Sequence
  Primer
aggtgactgggaggacttgaggc

SEQ ID NO: 11 - DNA, 20 bp, Artificial Sequence
  Primer
ccccagcggcaccatgtctg

SEQ ID NO: 12 - DNA, 21 bp, Artificial Sequence
  Primer
tgggccaaccgggtgaaggaa

SEQ ID NO: 13 - DNA, 23 bp, Artificial Sequence
  Primer
ttcagcaagaagaacaaggacaa

SEQ ID NO: 14 - DNA, 22 bp, Artificial Sequence
  Primer
ggttgaagaagtcctcctaagc

SEQ ID NO: 15 - DNA, 20 bp, Artificial Sequence
  Primer
tacacaccctttggaagtgg

SEQ ID NO: 16 - DNA, 20 bp, Artificial Sequence
  Primer
caaagcgtaatttcaggggg

SEQ ID NO: 17 - DNA, 25 bp, Artificial Sequence
  Primer
tgtccatttcatcacaaatcccttg

SEQ ID NO: 18 - DNA, 23 bp, Artificial Sequence
  Primer
gtggtatttccatccatcgggtc

SEQ ID NO: 19 - DNA, 21 bp, Artificial Sequence
  Primer
ttggaggtgttttgactgctt

SEQ ID NO: 20 - DNA, 23 bp, Artificial Sequence
  Primer
acaagtggataaggtcgatgttg

SEQ ID NO: 21 - DNA, 20 bp, Artificial Sequence
  Primer
tcagccctggcccagatccc

SEQ ID NO: 22 - DNA, 20 bp, Artificial Sequence
  Primer
acagctgctcccctggcctt

SEQ ID NO: 23 - DNA, 20 bp, Artificial Sequence
  Primer
actgaatccagaacactgca

SEQ ID NO: 24 - DNA, 20 bp, Artificial Sequence
  Primer
tgcagtcaatgcatctttca

SEQ ID NO: 25 - DNA, 19 bp, Artificial Sequence
  Primer
tggcacccagcacaatgaa

SEQ ID NO: 26 - DNA, 25 bp, Artificial Sequence
  Primer
ctaagtcatagtccgcctagaagca

SEQ ID NO: 27 - DNA, 20 bp, Artificial Sequence
  Primer
tgatttctctggcctcttgg

SEQ ID NO: 28 - DNA, 20 bp, Artificial Sequence
  Primer
ttgtcctttcttcctcctgg

SEQ ID NO: 29 - DNA, 23 bp, Artificial Sequence
  Primer
gagtacaaccttcttgcagctcc

SEQ ID NO: 30 - DNA, 22 bp, Artificial Sequence
  Primer
ccttctgacccatacccaccat

(19) United States
(12) Patent Application Publication
(10) Pub. No.: US 2024/0108590 A1
(43) Pub. Date: Apr. 4, 2024

(54) MAINTENANCE OF PROGRAMMED CHRONIC LIVER INJURY OF FAH GENE-DEFICIENT ANIMALS AND APPLICATION THEREOF IN PREPARATION OF HETEROLOGOUS LIVER MODELS

(71) Applicant: Jiangsu University, Zhenjiang, Jiangsu (CN)

(72) Inventors: Yunwen Zheng, Jiangsu (CN); Jianyun GE, Zhejiang (CN); Liping Liu, Jiangsu (CN)

(73) Assignee: Jiangsu University, Zhenjiang, Jiangsu (CN)

(21) Appl. No.: 18/039,105

(22) PCT Filed: Nov. 29, 2021
(86) PCT No.: PCT/CN2021/134077

§ 371 Date: Nov. 21, 2023

(30) Foreign Application Priority Data
Nov. 30, 2020 (CN) 202011379101.6

(51) Int. Cl. A61K 31/122; A01K 67/027; A61K 31/407; A61P 1/16

(52) U.S. Cl. CPC A61K 31/122; A01K 67/0271; A61K 31/407; A61P 1/16; A01K 2207/12; A01K 2227/105; A01K 2267/03

(57) ABSTRACT

Provided in the present invention are the maintenance of the programmed chronic liver injury of FAH gene-deficient animals and an application thereof in the preparation of heterologous liver models. The present invention discloses a programmed NTBC controlling (NTBC^{P}) scheme with a fixed cycle period, the scheme enabling animal models to survive for a long time in a state of chronic liver injury, and being capable of achieving a high heterologous replacement rate after a heterologous hepatocyte transplantation is performed.

### TECHNICAL FIELD

The disclosure belongs to the technical field of biotechnology and animal model preparation. More specifically, the disclosure relates to a method for maintaining programmed chronic liver injury of Fah gene-deficient animals and application thereof in preparation of heterologous liver models.

### TECHNICAL BACKGROUND

Liver is a vital site for the metabolism and clearance of drugs in human and also a host of multiple specific pathogens. Due to differences between species, these human liver-specific functions and characteristics are difficult to be effectively recapitulated in other animals. Currently, humanized liver has been successfully achieved in mice by transplanting primary human hepatocytes (PHH) (M Dandri, et al., Hepatology. 33(4): 981-988, 2001; DF Mercer, et al., Nat Med. 7(8):927-933, 2001; C Tateno, et al., Am J Pathol. 165(3):901-912, 2004). It can be applied on the accurately prediction of human liver-specific drug metabolism and pathogen infection. In addition, humanized animal models can also be used as bioreactors to produce large amounts of functional human hepatocytes for various purposes, such as “artificial liver systems” that require large amounts of hepatocytes (H Azuma, et al., Nat Biotechnol. 25(8):903-910, 2007; E Michailidis, et al., Proc Natl Acad Sci USA. 117(3):1678-1688, 2020). The research and development achievements in the past ten years have successfully promoted the efficient and stable preparation of humanized liver mouse models (H Azuma, et al., Nat Biotechnol. 25(8):903-910, 2007; C Tateno, et al., PLoS One. 10(11):e0142145, 2015; E M Wilson, et al., Stem Cell Res. 13(3 Pt A):404-412, 2014; M Hasegawa, et al., Biochem Biophys Res Commun. 405(3):405-410, 2011; KD Bissig, et al., J Clin Invest. 120(3):924-930, 2010; ML Washburn, et al., Gastroenterology. 140(4):1334-1344, 2011). However, because of size limitations, mice cannot provide sufficient amounts of biological samples, such as blood and bile for pharmacological analyses and hepatocytes for regenerative medicine researches (K Yoshizato, et al., Expert Opin Drug Metab Toxicol. 9(11):1419-1435, 2013).

Compared with mice, rats are at least 10 times larger than mice in size, and are also similar to humans in terms of physiology and pathology (P M Iannaccone, et al., Dis Model Mech. 2(5-6):206-210, 2009). US FDA guidelines also recommend the use of rats for preclinical studies, particularly drug metabolism and toxicology (H J Jacob, et al., Nat Rev Genet. 3(1):33-42, 2002). In addition, more than 10⁹ human hepatocytes can be provided theoretically if humanized liver replacement is complete in rats. Therefore, the construction of humanized livers in rat models is more important than in mice, especially in the fields of drug development and regenerative medicine research. However, the development of liver-humanized rats has been the greatly hindered due to the lack of effective rat models for human-derived hepatocyte xeno-transplantation. At the same time, practical experience in implementing liver humanization in large animals other than mice is also insufficient currently.

The inventors have previously participated in FRG (Fah^{-/-}Rag2^{-/-}IL2rg^{-/-}) rats with fumaryl acetoacetate hydrolase (Fah) gene deficiency combined with severe immunodeficiency (Chinese patent pending, application number: 201810621840.8), the rat was used to construct humanized liver, and it was confirmed that this rat model has the potential to construct humanized liver. However, for long-term maintenance of transplanted rats, it is necessary to further explore an effective liver injury regulation mode to achieve a stable and efficient level of humanized replacement.

### SUMMARY OF THE DISCLOSURE

The purpose of the disclosure is to provide a method for maintaining non-acute lethal and sustainable chronic liver injury in Fah gene-deficient rats, and use this method to realize stable expansion of human hepatocytes in rats, and finally obtain heterologous liver with high replacement rate.

The purpose of the disclosure is also to provide a system or kit, wherein it can be used for preparing a Fah gene-deficient rat model and maintaining chronic liver injury and can be used for preparing a Fah gene-deficient rat model and maintaining chronic liver injury.

In the first aspect, the present disclosure provides a method for maintaining chronic liver injury in a Fah gene-deficient (Fah^{-/-}) animal model, wherein the method comprises administering Nitisinone (NTBC) to the Fah gene-deficient animal model according to the following scheme: (1) administering a low dose of Nitisinone every day for 3-12 days, wherein the low dose is 0.005-0.1 mg/kg animal body weight/day; (2) administering a high dose of Nitisinone every day for 2-6 days, wherein the high dose is 0.25-1.5 mg/kg animal body weight/day; (3) repeat the cycle of step (1) and (2).

In the first aspect, the present disclosure provides a method for preparing a heterologous liver transplantation animal model, wherein the animal model has a heterologous liver, the method comprises: (1) administering a low dose of Nitisinone every day for 3-12 days, wherein the low dose is 0.005-0.1 mg/kg animal body weight/day; (2) administering a high dose of Nitisinone every day for 2-6 days, wherein the high dose is 0.25-1.5 mg/kg animal body weight/day; (3) repeat the cycle of step (1) and (2): and on the 3rd to 30th days after the initiation of the above scheme, the animals were transplanted with heterologous hepatocytes.

In a preferred embodiment of the disclosure, before carrying out the scheme, it further comprises: pretreating the animal with Retrorsine; preferably, the dose of Retrorsine is 10-50 mg/kg animal body weight; more preferably, the dose of Retrorsine is 2040 mg/kg animal body weight; further preferably, the dose of the Retrorsine is 25-35 mg/kg animal body weight.

In another preferred embodiment of the disclosure, in step (1), administering a low dose of Nitisinone every day for 4-10 days; preferably, administering a low dose of Nitisinone every day for 5, 6, 7, 8 or 9 days.

In another preferred embodiment of the disclosure, in step (1), the low dose is 0.008-0.08 mg/kg animal body weight/day; preferably, the low dose is 0.01-0.05 mg/kg animal body weight/day; such as 0.015-0.04 mg/kg animal body weight/day, or 0.015-0.035 mg/kg animal body weight/day; more specifically, such as 0.02 mg/kg animal body weight/day, 0.03 mg/kg animal body weight/day.

In another preferred embodiment of the disclosure, in step (2), administering a high dose of Nitisinone every day for 3-5 days.

In another preferred embodiment of the disclosure, in step (2), the high dose is 0.3-1.2 mg/kg animal body weight/day; preferably, the high dose is 0.35-1 mg/kg animal body weight/day; such as 0.35-0.85 mg/kg animal body weight/day, more specifically, such as 0.4, 0.5, 0.6, 0.7, 0.8 mg/kg animal body weight/day.

In another preferred embodiment of the disclosure, the animal is a mammal that is at least twice (such as 2-2000 times, more specifically such as 5, 10, 15, 20, 50, 100, 200, 500 times) larger in size than a mouse; preferably, the animal comprises an animal selected from the group consisting of: rat, rabbit, monkey, pig, guinea pig, dog; preferably, the animal is an animal where in the interleukin 2 receptor gamma gene and the recombination activating gene 2 are destroyed (Rag2^{-/-}IL2rg^{-/-}).

In another preferred embodiment of the disclosure, the heterologous hepatocytes comprise (but not limited to): hepatocytes that belongs to different species from the animal model and derived from human, mouse, pig, monkey or dog; preferably, the heterologous hepatocytes comprise (but not limited to): (a) primary hepatocytes; (b) hepatic stem/progenitor cells, hepatocytes or hepatocyte-like cells, endoderm cells, etc, induced and differentiated from induced pluripotent stem cells (iPSC) and embryonic stem cells (ESC); (c) hepatic stem/progenitor cells, hepatocytes or hepatocyte-like cells induced and differentiated from stem cells of endoderm and other germ layers; (d) hepatic stem/progenitor cells, hepatocytes or hepatocyte-like cells derived from direct transdifferentiation of adult or fetal stem/progenitor cells or hepatocytes and other somatic cells; (f) hepatic stem/progenitor cells, hepatocytes or hepatocyte-like cells after in vitro induced expansion of cells derived from (a)-(d); (g) liver organoids constructed from cells derived from (a)-(f).

In another preferred embodiment of the disclosure, the cycle of (3) is performed until the animal model is applied or the life of the animal model ends.

In another preferred embodiment of the disclosure, the heterologous liver is a liver with a high heterologous repopulation rate.

In another preferred embodiment of the disclosure, the high heterologous repopulation rate (heterologous replacement rate) is the heterologous repopulation rate of more than 25%, preferably more than 28%/6, more preferably more than 30%. At the same time, with culture time of the recipient animal model passed, the repopulation rate increased continuously.

In another preferred embodiment of the disclosure, the heterologous hepatocytes are isolated from the liver of an organ donor, isolated from surgical resections or derived from induced pluripotent stem cells, embryonic stem cells, embryonic tissues and cells thereof, amniotic epithelial cells, monocytes or amniocytes.

In another preferred embodiment of the disclosure, the heterologous hepatocytes transplanted into the animal are isolated heterologous hepatocytes.

In another preferred embodiment of the disclosure, the Nitisinone is administered in drinking water.

In another preferred embodiment of the disclosure, the Nitisinone is administered in food.

In another aspect, the present disclosure provides a system for preparing a Fah gene-deficient animal model and maintaining chronic liver injury, comprising: an administration component or module 1, wherein it is set to administer a low dose of Nitisinone to animals every day for 3-12 days, wherein the low dose is 0.005-0.1 mg/kg animal body weight/day; and an administration component or module 2, wherein it is set to administer a high dose of Nitisinone to animals every day for 2-6 days, wherein the high dose is 0.25-1.5 mg/kg animal body weight/day.

In a preferred embodiment of the disclosure, the system for preparing a Fah gene-deficient animal model and maintaining chronic liver injury comprises a mechanical device or a computer system.

In another preferred embodiment of the disclosure, the device can also be used for preparing a heterologous liver animal model with a heterologous liver.

In another preferred embodiment of the disclosure, the device also comprises: an administration component or module 3, wherein it can administer Retrorsine to animals.

In another preferred embodiment of the disclosure, the device is set to administer Retrorsine at 10-50 mg/kg animal body weight.

In another preferred embodiment of the disclosure, the device delivers the dose of drugs through a computer program and delivers to the animal by mechanical devices.

In another aspect, the present disclosure provides a kit for preparing a Fah gene-deficient animal model and maintaining chronic liver injury, wherein it comprises: container group 1, comprising a container and a low dose of Nitisinone in the container, wherein the low dose is 0.005-0.1 mg/kg animal body weight; preferably, the number of containers in the container group 1 is 3-12; and container group 2, comprising a container and a high dose of Nitisinone in the container, wherein the high dose is 0.25-1.5 mg/kg animal body weight; preferably, the number of containers in the container group 2 is 2-6.

In another preferred embodiment of the disclosure, the kit also comprises: container 3, and a Retrorsine in the container; preferably, the dose of Retrorsine is 1.2-10 mg.

In a preferred embodiment of the disclosure, the container group 1 and container group 2 and preferably also container group 3 are used as a complete administration cycle of an animal (a single “low dose-high dose” is a cycle).

In another preferred embodiment of the disclosure, the container group 1 and container group 2 and preferably also container group 3 are used as two or more complete administration cycles of an animal, or one, two or more administration cycles of multiple animals.

In another preferred embodiment of the disclosure, in continuous animal preparation, a set of kits can be used, for example, the set of kits contain 2-200 kits, such as 5, 10, 15, 20, 30, 40, 50, 60, 80, 100, 150 kits.

In another aspect, the present disclosure provides a use of low-dose Nitisinone, high-dose Nitisinone and Retrorsine, for: maintaining chronic liver injury in a Fah gene-deficient animal model; or preparing an administration group and a set of kits for maintaining chronic liver injury in a Fah gene-deficient animal model; wherein, the low dose is 0.005-0.1 mg/kg animal body weight/day, the high dose is 0.25-1.5 mg/kg animal body weight/day.

In another aspect, the present disclosure provides a use of low-dose Nitisinone, high-dose Nitisinone and Retrorsine, for: preparing a heterologous liver animal model, wherein the model has a heterologous liver; or preparing an administration group and a set of kits for the preparation of a heterologous liver animal model; wherein, the low dose is 0.005-0.1 mg/kg animal body weight/day, the high dose is 0.25-1.5 mg/kg animal body weight/day.

Other aspects of the present invention will be apparent to those skilled in the art based on the invention herein.

### BRIEF DESCRIPTION OF THE DRAWINGS

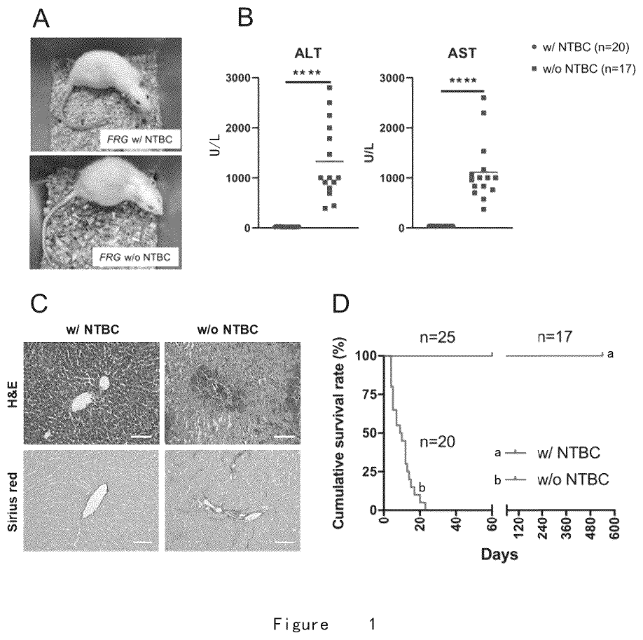

FIG. 1. NTBC-mediated liver injury in FRG rats.
A. Images of FRG rats living with NTBC supply (w/) and without NTBC supply (w/o).
B. Serum ALT and AST levels in healthy FRG rats with NTBC supply and moribund FRG rats after NTBC withdrawal. ****P<0.0001.
C. Results of HE and Sirius red staining of livers from FRG rats with NTBC supply and FRG rats after NTBC withdrawal. Scale bar, 100 μm.
D. Survival curve of FRG rats with and without NTBC supply.

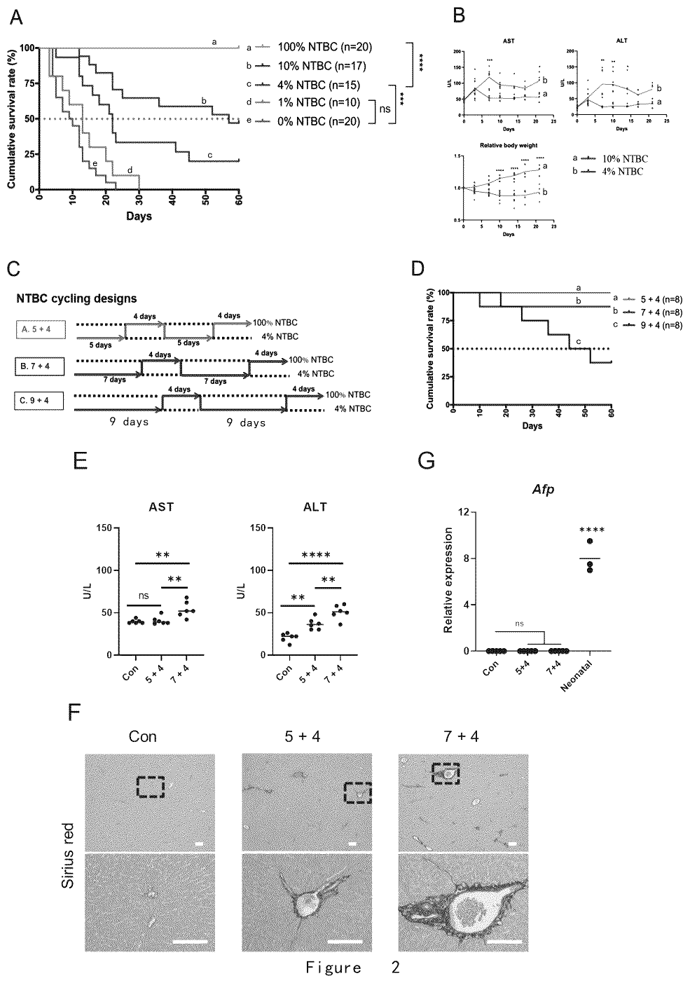

FIG. 2. Establishment of a rat model of chronic liver injury.
A. Survival curve of FRG rats supplied with different concentrations of NTBC. ***P<0.001, ****P<0.0001; ns, not statistically significant.
B. Levels of AST and ALT and body weight in rats supplied with 4% and 10% NTBC. *P<0.05, **P<0.01, ***P<0.001. ****P<00001.
C. Schematic outline of programmed NTBC controlling scheme with a fixed cycle period.
D. Survival curve of FRG rats under the controlling of three different programmed NTBC (5+4, 7+4 and 9+4).
E. Levels of ALT and AST in rats under the controlling of programmed NTBC (5+4 and 7+4). **P<0.01, ****P<0.0001; ns, not statistically significant.
F. Sirius red staining shows the degree of fibrosis in rat livers under the controlling of programmed NTBC (5+4 and 7+4). Scale bar, 100 μm.
G. Expression levels of Afp gene in rat livers under the controlling of programmed NTBC (5+4 and 7+4). ****P<0.0001; ns, not statistically significant.

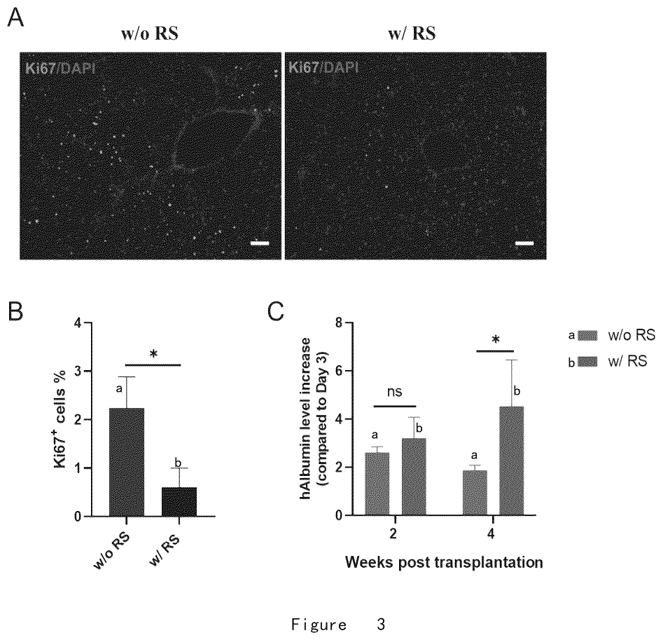

FIG. 3. Retrorsine (RS) pretreatment and repairing regeneration of transplanted rat hepatocytes.
A. Ki67 staining results in rat livers with (w/) or without (w/o) RS pretreatment. Scale bar, 100 μm.
B. Statistical analysis of the proportion of proliferative cells in rat livers with (w/) or without (w/o) RS pretreatment. *P<0.054
C. Levels of human albumin secretion in rats with (w/) or without (w/o) RS pretreatment within 4 weeks after PHH transplantation. *P<0.05.

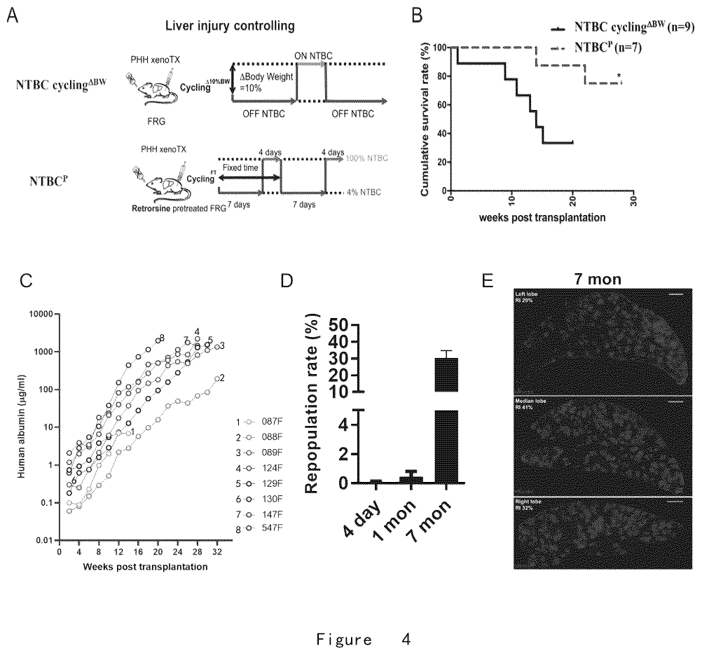

FIG. 4. Establishment of a rat model of chronic liver injury.
A. Illustration of the controlling of NTBC^{P} and NTBC^{ΔBW} in liver injury.
B. Survival curve of PHH-transplanted FRG rats under the controlling of NTBC^{P} and NTBC^{ΔMW}.
C. The dynamic change of human albumin concentration in serum of transplanted rats.
D. The ratio of repopulated human hepatocytes in FRG rat livers after transplantation for 4 days, 1 month or 7 months.
E. Staining results of human-derived nuclei (Nuclei) sections of liver lobes (the median, left and right liver lobes) in rats 7 months after transplantation. Scale bar, 2 mm.

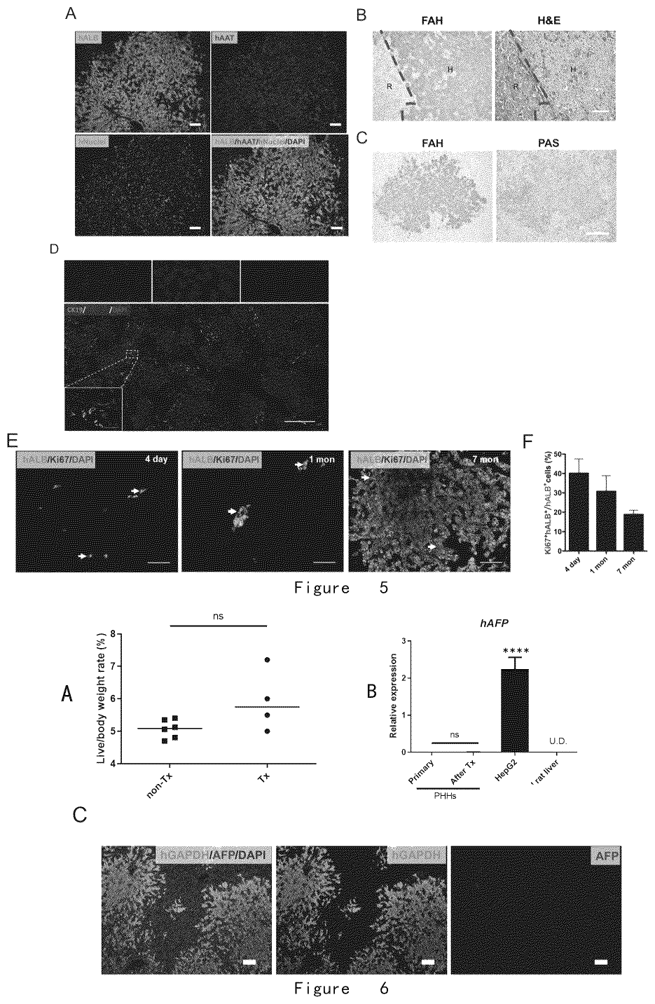

FIG. 5. Identification of humanized livers.
A. Human hepatocytes were identified by counterstaining results of hALB, hAAT and hNuclei. Scale bar, 100 μm.
B. FAH and HE staining of serial sections from humanized livers. R, rat hepatocytes; H, human hepatocytes. Scale bar, 100 μm.
C. FAH and PAS staining of serial sections from humanized livers. Scale bar, 100 μm.
D. CK19 and hNuclei counterstaining results of sections from humanized livers. Scale bar, 1 mm (in enlarged figure: 100 μm).
E. Ki67 and hALB counterstaining results after transplantation for 4 days, 1 month or 7 months. Scale bar, 100 μm.
F. Proportion of Ki67⁺ proliferative cells in hALB⁺ human hepatocytes at different time points after transplantation.

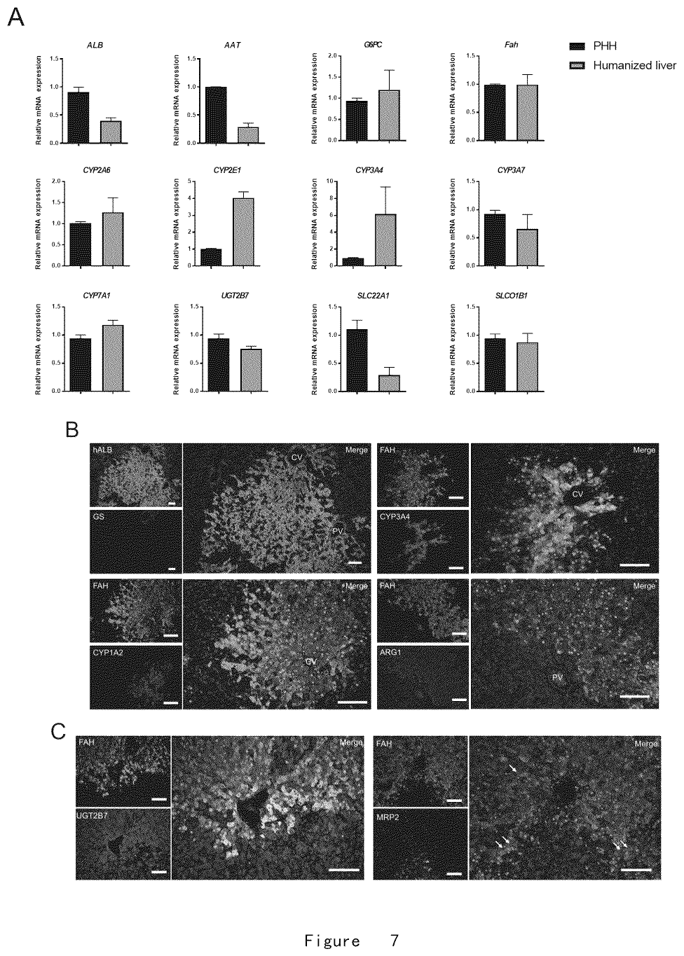

FIG. 6. Detection of humanized liver tumorigenesis.
A. Liver/body weight rate of PHH-transplanted and non-transplanted FRG rats, ns, not statistically significant.
B. Expression of hAFP gene in FRG rat livers with PHH-transplantation and non-transplantation. HepG2 HCC cell line was used as a positive control. ****P<0.0001; ns, not statistically significant; U.D., undetected.
C. Immunostaining results of AFP protein in FRG rat livers transplanted with PHH, hGAPDH is an internal reference for human cells. Scale bar, 100 μm.

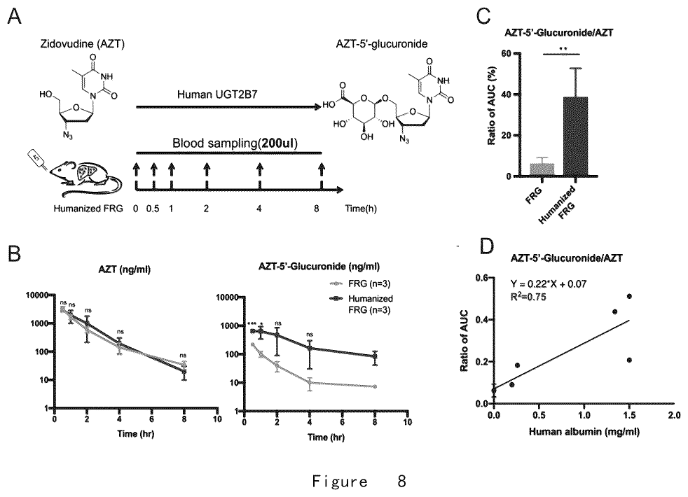

FIG. 7. Humanized liver metabolism-related gene and protein expressions.
A. Comparison of gene expression of mature hepatic markers, phase I, phase II metabolizing enzymes, and transporter in PHHs from humanized livers and donors by qPCR analysis. PHH, primary human hepatocytes.
B. Immuno-costaining results of ALB. FAH, CYP3A4, CYPIA2, ARG1, CV, central vein; PV, portal vein; Scale bar, 100 μm.
C. Immuno-costaining results of FAH. UGT2B7 and MRP2. Scale bar, 100 μm.

FIG. 8. UGT2B7 metabolism in humanized livers.
A. Schematic outline of the analysis of UGT2B7 metabolism of AZT. The rats were orally administered with AZT (15 mg/kg) and samples were collected at indicated time points for testing.
B. Time-dependent change in the concentration of AZT and AZT-5′-glucuronide (GAZT) in rats with liver humanization and rats in the control group. ***P<0.001; ns, not statistically significant.
C. Ratio of AUC for AZT-5′-glucuronide/AZT in rats with liver humanization and rats in the control group. **P<0.01.
D. The correlation between the ratios of AUC (AZT-5′-glucuronide/AZT) and human albumin secretion levels in the liver humanized rats.

### DETAILED DESCRIPTION

In view of the technical defect that it is still difficult to obtain an animal model that can maintain long-term survival and has a high repopulation rate of heterologous liver in the art, by in-depth researches, the inventors revealed a Nitisinone (2-(2-nitro-4-trifluoromethylbenzoyl)-1.3 cyclohexanedione. NTBC) programmed NTBC controlling (NTBC^{P}) scheme with a fixed cycle period. The scheme of the present disclosure can realize long-term survival of animals in the state of chronic liver injury, and can realize high heterologous replacement rate after heterologous hepatocyte transplantation.

As used in the present disclosure, the “deficiency of the fumaryl acetoacetate hydrolase gene (Fah^{-/-})”. “deficiency of the recombination activation gene 2 (Rag2^{-/-})” or “deficiency of the interleukin 2 receptor gamma gene (IL2rg^{-/-})” means that Fah. Rag2 or IL2rg is disrupted, comprising disruption by means such as gene knockout, gene editing, homologous recombination or site-directed mutagenesis, etc. In some aspects of the present disclosure, the “deficiency” also comprises significant reduction in gene/protein expression. e.g., about 80%, about 90%, about 95%, or about 99% reduction in gene/protein expression.

As used in the present disclosure, the animal is a mammal. Preferably, the animal is a mammal that is at least twice (such as 2-2000 times) larger in body size than a mouse; more preferably, the animal comprises an animal selected from the group consisting of: rat, rabbit, pig, guinea pig, dog and non-human primates such as monkey, orangutan; preferably, the animal is a Fah^{-/-}Rag2^{-/-}IL2rg^{-/-} animal. Most preferably, the animal is a rat.

As used in the present disclosure, the “heterologous” is “nonendogenous”, which refers to the relationship between two types of hepatocytes from different species, or the relationship between cells from different sources and animal recipients. For example, if the combination of a cell and an animal recipient does not normally occur in nature, the cell is heterologous to the animal recipient. The “heterologous” may also be referred to as “exogenous”. The “heterogeneity” refers to a process of heterologous cells to colonize, grow, amplify, and exert functions in the liver of a recipient animal.

As used in the present disclosure, the “heterologous repopulation rate” refers to a ratio of heterologous cells in total hepatocytes of the recipient animal after the heterologous cells colonize, grow and amplify in the liver of the recipient animal.

Preparation of Animal Models

FAH enzyme is a key enzyme in liver tyrosine metabolism pathway. In animal models, deficiency of the Fah gene can cause the accumulation of tyrosine metabolic intermediate succinyl, which can lead to the death of hepatocytes after reaching a certain level. However, NTBC supplementation in daily drinking water can prevent the accumulation of toxic metabolic intermediates by inhibiting the tyrosine metabolic pathway, and play a role in rescuing liver injury. In the art, although a cyclic regulation strategy of NTBC withdrawal/administration based on stepped withdrawal or body weight change has been applied to human hepatocyte transplantation in Fah-deficient mice. However, in preliminary tests of present inventors, it was found that this regulation strategy can only support a low proportion (less than 10%) of human hepatocyte repopulation in Fah-deficient rats, and cannot be applied to the construction of highly humanized livers. The inventors have found that Fah-deficient rats are more sensitive to liver injury caused by NTBC withdrawal than mice with isogenic gene deficiency; and the body mass index is too seriously lagged to hardly rescue in time, resulting in extremely high mortality. The window for rescue after transplantation of mature hepatocytes is too short; thus, stable and efficient humanized replacement rates cannot be obtained. Therefore, the present inventors have endeavored to find a solution to this problem.

On the basis of in-depth researches and analyses, the inventors revealed a novel method for chronic liver injury with good operability, high controllability, non-acute lethality and sustainable maintenance. The method of the disclosure can avoid early death of model rats after transplantation of heterologous hepatocytes, create an intrahepatic environment and a sufficient expansion window period that are favorable for the colonization and survival of heterologous hepatocytes or hepatocyte-like cells in FRG rats, and construct heterologous livers with high replacement rates and high survival rates. This method can also be applied to the transplantation of heterologous hepatocytes or hepatocyte-like cells in Fah-deficient large animal models other than rats, such as rabbits, pigs, guinea pigs, dogs, non-human primates such as monkeys, etc.

Based on new findings of inventors, the present disclosure provides a method for maintaining chronic liver injury in a Fah gene-deficient animal model (NTBC^{P}), wherein the method comprises administering NTBC to the Fah gene-deficient animal model according to the following scheme: (1) administering a low dose of Nitisinone every day for 3-12 days, wherein the low dose is 0.005-0.1 mg/kg animal body weight/day; (2) administering a high dose of Nitisinone every day for 2-6 days, wherein the high dose is 0.25-1.5 mg/kg animal body weight/day; (3) repeat the cycle of step (1) and (2). According to actual needs, the scheme can be carried out until the animal model is applied or the life of animal model is over.

In view of individual differences among different animals, the daily intake of NTBC fluctuates to a certain extent under oral administration, so the low dose or high dose of the present disclosure is based on the average intake of animals. Specifically, for the low-dose administration stage, when 0.005-0.1 mg/kg of animal body weight NTBC is administered on average every single day, 5 mg/L NTBC is set as 100% NTBC, corresponding NTBC drug concentration can be 0.665-13.33%. Specifically, for the high-dose administration stage, when 0.25-1.5 mg/kg of animal body weight NTBC is administered on average every single day, 5 mg/L NTBC is set as 100% NTBC, corresponding NTBC drug concentration can be 33.33%-200%.

Fah gene-deficient animals can control liver injury by artificially regulating NTBC supplement, thereby creating conditions for Fah wild-type hepatocyte transplantation or xenograft transplantation. Therefore, on the basis of maintaining chronic liver injury of the Fah gene-deficient animal model, the present disclosure further provides a method for preparing a heterologous liver animal model, comprising: using the steps (1) to (2) above, and, on the 3rd to 30th day (such as 4th, 5th, 6th, 8th, 10th, 12nd, 15th, 18th, 20th, 22nd, 25th, 28th days) after the initiation of the above scheme, the animals were transplanted with human primary hepatocytes.

In a preferred embodiment of the present disclosure, the Fah gene-deficient animal is a Fah^{-/-}Rag2^{-/-}IL2rg^{-/-} animal. Fah is an enzyme with 419 amino acids, its gene length is 22586 bp, with 14 exons and 13 introns; its GenBank accession number is NM_017181.2. Rag2 is an enzyme with 527 amino acids, its gene length is 8297 bp, with 3 exons and 2 introns; its GenBank accession number is NM_001100528.1. IL2rg is an enzyme with 368 amino acids, its gene length is 7281 bp, with 12 exons and 11 introns; its GenBank accession number is NM_080889.1. Loss of Fah gene can lead to inducible liver injury in animals. Knockout of Rag2 gene can lead to the loss of T and B cells in animals, and knockout of IL2rg gene can lead to the loss of NK cells and decrease of T and B cells in animals. Rag2 and IL2rg knockout individually and together can obtain immunodeficient animals, while Rag2 and IL2rg knockout at the same time have the most severe immunodeficiency and are suitable for human hepatocyte transplantation.

In the present disclosure, NTBC can be administered to animals in a variety of ways. In some preferred embodiments, oral administration is used, comprising mixing NTBC with drinking water, or mixing NTBC with food of animals.

In the present disclosure, the heterologous hepatocytes may comprise: (a) primary hepatocytes; (b) hepatic stem/progenitor cells, hepatocytes or hepatocyte-like cells, endoderm cells, etc. induced and differentiated from induced pluripotent stem cells (iPSC) and embryonic stem cells (ESC); (c) hepatic stem/progenitor cells, hepatocytes or hepatocyte-like cells induced and differentiated from stem cells of endoderm and other germ layers; (d) hepatic stem/progenitor cells, hepatocytes or hepatocyte-like cells derived from direct transdifferentiation of adult or fetal stem/progenitor cells or hepatocytes and other somatic cells; (f) hepatic stem/progenitor cells, hepatocytes or hepatocyte-like cells after in vitro induced expansion of cells derived from (a)-(d); (g) liver organoids constructed from cells derived from (a)-(f). The heterologous primary hepatocytes can be isolated from the liver of an organ donor, isolated from surgical resections or derived from induced pluripotent stem cells, embryonic stem cells, embryonic tissues and cells thereof, amniotic epithelial cells, monocytes or amniocytes.

In the present disclosure, heterologous hepatocytes for transplantation into recipient animals can be isolated from livers by any method known in the art. Livers can be mechanically or enzymatically digested to provide a single cell suspension, or whole livers can be used. For example, hepatocytes can be isolated from donor tissues by conventional collagenase perfusion followed by low speed centrifugation; hepatocytes can then be purified by mesh filtration followed by density gradient centrifugation. As a different option, other methods for enriching hepatocytes can also be used, such as flow cytometry, magnetic bead separation, density gradient centrifugation, or any other method known in the art. Expanded human hepatocytes can be collected from recipient animal livers using similar hepatocyte isolation methods. In addition to obtaining heterologous hepatocytes from organ donors or hepatectomy tissue, the cells used for engraftment can also be human hepatocytes that have colonized and expanded after transplantation into the recipient animal or precursor cells, progenitor cells of hepatocytes and other hepatocyte-like cells expanded in vitro.

As a preferred embodiment of the present disclosure, before carrying out the scheme (NTBC^{P}), the method further comprises: pretreating the animals with Retrorsine (RS). Before the development of the NTBC^{P} scheme, the inventors also tried the combination of Retrorsine and partial hepatectomy to treat animals. However, due to limitations of the animal model for hardly regulating liver injury, the level of detected human albumin secretion was only 90 μg/ml. In humanized animals, only when the humanized repopulation rate exceeds 30% or the albumin secretion level of 1 mg/ml can accurately simulate human drug metabolism. Therefore, the inventors have developed an optimized NTBC^{P} scheme using Fah-deficient and immunodeficient rats as a model. After transplanted for 7 months, humanized repopulation rate can be greatly increased to more than 30% and the level of albumin secretion can reach to more than 2 mg/ml.

As a preferred embodiment of the present disclosure, the dose of Retrorsine is 10-50 mg/kg animal body weight; more preferably, the dose of Retrorsine is 2040 mg/kg animal body weight; further preferably, the dose of Retrorsine is 25-35 mg/kg animal body weight. In a specific example of the present disclosure, it was demonstrated that the proportion of Ki67 positive cells was significantly lower in livers of RS pretreated animals. The level of human albumin (hAlbumin, ALB) secretion is an important indicator to evaluate the level of repopulation and expansion of heterologous hepatocytes in vivo. By comparison, it was found that the level of albumin secretion in animals pretreated with RS after transplantation of heterologous hepatocytes increased significantly faster, indicating that the combination of RS pretreatment and NTBC programming could synergistically promote the repopulation and expansion of heterologous hepatocytes in vivo.

In a specific embodiment of the present disclosure, a preferred embodiment is provided, comprising: 2 weeks before transplantation of human-derived hepatocytes or hepatocyte-like cells, Retrorsine intraperitoneal injection of 30 mg/kg animal body weight is administered to Fah-deficient rats. One week before transplantation, the NTBC concentration in the daily water supply was reduced from a normal maintaining concentration (5-8 mg/L) to a low concentration (0.2 mg/L), followed by the programmed NTBC scheme, according to a cycle of 0.2 mg/L×7 days+5 mg/L×4 days (11 days in one cycle period), with NTBC added to the daily drinking water.

Using the method of the present disclosure, a heterologous liver with high heterozygous repopulation rate can be obtained. The heterologous repopulation rate can be more than 25%, preferably more than 28%, more preferably more than 32%; The repopulation rate will further increase with the increase of animal culture time.

The technical solution of the present disclosure has very significant technical effects, comprising but not limited to: (1) maintaining long-term chronic liver injury of Fah-deficient animals, achieving non-acute lethal and sustainable chronic liver injury, avoiding early death of animal models after transplantation of heterologous hepatocytes or hepatocyte-like cells, and having a high rate of animal survival. (2) creating an intrahepatic environment that is conducive to the colonization and survival of heterologous hepatocytes or hepatocyte-like cells in animal models, providing a long enough expansion window in vivo and constructing heterologous livers with high replacement rate and high survival rate; (3) having convenient and controllable methods for operation and management, with the device or computer system for automatic preparation of animal models be developed, realizing large-scale preparation of animal models.

The Applications

In the present disclosure, the animal model for heterologous hepatocyte transplantation can be used for a variety of researches and therapeutic purposes, comprising but not limited to the following applications:

Animal chronic liver injury model: Because chronic liver injury can be stably induced in Fah-deficient animals by the present disclosure, a chronic liver disease model mainly represented by liver fibrosis and liver cirrhosis can be established and used for disease mechanism and drug researches.

Preclinical drug tests in vivo: the animal model with high liver heterogeneity constructed by the present disclosure is expected to be used for preclinical drug metabolism, toxicity, drug efficacy tests, and human hepatitis virus infection experiments in vivo. It is not only similar to humans in terms of physiological aspect, but also used for long-term sampling, testing and tracking and it is desirable for promoting new drug developments. The disclosed animal model can be used for studying multiple liver diseases, including HCC, hepatic carcinoma, and liver cirrhosis and so on. The disclosed animal model can be used as a model for liver disease caused, for example, by exposure to toxins, infectious diseases or malignancies, or by genetic defects. Examples of inherited liver diseases suitable for drug research using the animal model of the present disclosure include but are not limited to hypercholesterolemia, hypertriglyceridemia, hyperoxaluria, phenylketonuria, glycogen storage disease and some inborn errors of metabolism. The disclosed model system can be used for better understanding specific liver diseases, as well as identifying agents that can prevent, delay or reverse the diseases. In another optional embodiments, the disclosed animal model can be used as a model for liver diseases caused by exposure to toxins. The disclosed animal model can also be used for sceening candidate vaccines with the ability of preventing or reducing infections by hepatotropic pathogens. In present disclosure, the type of drug candidates used for drug testing is not particularly limited, and can be obtained from various sources, including synthetic or natural compound libraries. For example, there are various ways for randomly and directly synthesizing a wide variety of organic compounds and biomolecules, including a way of expressing random oligonucleotides and oligopeptides; or a way of Natural Compound Library that are available or easy to produce bacteria, fungi, plants and animal extracts. In addition, libraries and compounds produced by natural or synthetic methods can be readily modified by conventional chemical, physical and biochemical methods and can be used to generate combinatorial libraries. Directed or random chemical modifications (e.g., acylation, alkylation, esterification, amidation, etc.) of known pharmacological agents can be made to generate structural analogs.

Heterologous (Human) Hepatocytes for Expansion Transplantation treatment: The method of the present disclosure allows heterologous (e.g., human) hepatocytes be expanded in and collected from a recipient animal as a source of human hepatocytes for liver remodeling in a subject in need of liver remodeling therapy. Supported by the present disclosure, heterologous hepatocytes/hepatocyte-like cells can be expanded substantially in Fah-deficient animals. To develop organoids for transplantation and replacement provides an important support for clinical regenerative transplantation therapy in the future. Reconstruction of livers in patients by introduction of hepatocytes is a potential therapeutic option for patients with acute liver injury and can also be used as a temporary treatment prior to liver transplantation. Hepatocyte reconstruction can be used, for example, to introduce genetically modified hepatocytes for gene therapy, or to replace hepatocytes loosed by diseases, physical or chemical injuries, or tumors. Human hepatocytes can be collected from recipient animals using a number of techniques known in the art; human hepatocytes collected from recipient animals can be separated from non-human cells or other impurities (such as tissues or cell debris). The present disclosure is expected to solve the serious problem of insufficient donors for liver organ and liver cell transplantation.

Gene therapy studies: Hepatocytes expanded in and collected from the animal models established by the present disclosure can be used for assessing the effects of any pharmaceutical compound (e.g., small molecules, biologics, environmental and biological toxins or gene delivery systems) on human hepatocyte gene expressions. It can also be applied to study gene therapys and vectors. For example, the following parameters can be assessed: transduction efficiency of gene delivery vectors (including viral and non-viral vectors); integration frequency and location of genetic material (integration site analysis); functionality of genetic material (gene expression level, gene knockout efficiency); and side effects of genetic material (in vivo analysis of human hepatocyte gene expressions or proteomics). For example, the use of transfected hepatocytes in gene therapy of patients with familial hypercholesterolemia has been reported.

Personalized liver disease models and precision medicine: Personalized liver disease animal models are constructed by using iPSC-derived hepatocyte-like cells from the patents, and used for gene editing therapies of liver diseases ranging from liver fibrosis, liver cirrhosis, HCC. ICC. CCC to NASH, HBV, and viral and metabolic genetic defects such as HT1 and AAT, promoting the development of personalized targeted drugs and frontier novel therapeutic strategies.

In vitro test: After heterologous hepatocytes/hepatocyte-like cells are expanded in animals, they can be recovered and obtained by liver perfusion. Since they are not cultured in vitro, the expanded heterologous hepatocytes/hepatocyte-like cells in vivo can maintain the maximum consistency with the original cells in terms of properties and functions. When used in related in vitro tests such as drug tests and disease simulations, it can ensure the stability and repeatability of the results.

In addition, methods similar to the present disclosure can be used to determine defined concentrations and maintenance time of NTBC suitable for a specific species, establish a programmed NTBC with a fixed cycle period, create a targeted regulation of chronic liver injury and achieve continuous and stable repopulatation and expansion of heterologous hepatocytes/hepatocyte-like cells in vivo after transplantation.

Animal Model Preparation System/Kit

Based on the disclosed novel method, the present disclosure also provides a system for cultivating Fah gene-deficient animal models and maintaining chronic liver injury. In the present disclosure, the system can be a mechanical device, preferably an automatic mechanical device; it can also be a computer system, comprising various modules to facilitate the implementation of the method of the present disclosure.

As a preferred embodiment, the present disclosure provides a mechanical device, preferably an automatic mechanical device, comprising: an administration component 1, wherein it is set to administer a low dose of NTBC to animals every day for 3-12 days, the low dose is 2-10%; and, an administration component 2, which is set to administer a high dose of NTBC to animals every day for 2-4 days, the high dose is 70-100%. Preferably, it also comprises an administration component 3, wherein it can administer (e.g., inject) Retrorsine to animals. The above-mentioned three administration components can be constituted into an organic whole and organically integrated with the device for accommodating animals, thereby providing a convenient and automatic animal preparation system.

As a preferred embodiment, the present disclosure provides a computer control system, wherein it comprises a dosing module 1, comprising a control program capable of issuing instructions to cause a downstream system (such as a robotic mouth) operatively connected to (operated by) the module to administer a low dose of NTBC to animals daily for 3-12 days, the low dose is 2-10%; it also comprises a dosing module 2, wherein it comprises a control program capable of issuing instructions to cause a downstream system (such as a robotic mouth) operatively connected to (operated by) the module to administer a high dose of NTBC daily to animals for 2-6 days, the high dose is 70-100%. Preferably, it also comprises a dosing module 3, wherein it comprises a control program capable of issuing instructions to enable a downstream system (such as a syringe) operatively connected to (operated by) the module to administer Retrorsine to animals.

Based on the disclosed new method, the present disclosure also provides a kit (preferably for 1 cycle) for cultivating a Fah gene-deficient animal model and maintaining chronic liver injury, comprising: a container group 1, comprising a container and a low-dose NTBC in the container, the low dose is 0.005-0.1 mg/kg animal body weight/day; preferably, the number of containers in the container group 1 is 3-12 (preferably for 1 cycle); and container group 2, comprising a container and a high-dose NTBC in the container, the high dose is 0.25-1.5 mg/kg animal body weight/day; preferably, the number of containers in the container group 2 is 2-6 (preferably for 1 cycle). Preferably, the kit further comprises: a container 3 and a Retrorsine in the container. As a preferred embodiment, the container 3 can be a syringe. Multiple sets of kits can be set up for multiple cycles of NTBC. Meanwhile, in practice, according to the body weight of the animal, it is easy for those skilled in the art to add a specific amount of NTBC to the container.

Based on the overall disclosure of the present invention and the examples of embodiments, those skilled in the art can easily design or assemble.

The disclosure if further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (34 ed. Science Press, 2002) or followed the manufacturer's recommendation.

Materials and Methods

1. Animals

Fah^{-/-}Rag2^{-/-}IL2rg^{-/-} rats (FRG rats) were housed in the Experimental Animal Center of Jiangsu University.

2. Cells

Source information of the primary hepatocytes used for transplantation in the examples is shown in Table 1.

TABLE 1

| batch | | | | | cell |
|---|---|---|---|---|---|
| number | age | | gender | Cause of donor death | viability |
| QIE | 7 | months | male | hypoxia; | 86% |
| | | | | cardiovascular diseases | |
| XJL | 2 | years | female | hypoxia (drowning) | 84% |
| JFC | 1 | year | male | hypoxia; blunt force | 94% |
| | | | | trauma | |

All of the above were purchased from Bioreclamation IVT, USA.

3. Reagents
(1) RNA reverse transcription
The Revert Aid RT kit (Thermo Fisher Scientific) was used.
(2) Quantitative qPCR
The kit THUNDERBIRD SYBR qPCR Mix (Toyobo, Japan) was used.
(3) Measurement of human albumin secretion
The kit Human Albumin ELISA Quantitation Set (Bethyl Laboratory, USA) was used.
(4) HE and immunostaining
4% paraformaldehyde (Wako); methanol (Wako); acetone (Wako); Tween-20 (Wako); citric acid (Wako); H₂O₂(Wako); goat serum (Thermo Fisher Scientific); donkey serum (Jackson Lab. USA); Hematoxylin (Wako); Eosin (Wako); ABC Kit (Vector Laboratories, USA); Opal™ 4-Color Manual IHC Kit (PerkinElmer. USA); 4,6-Diamidino-2-phenylindole (DAPI. Thermo Fisher Scientific); FA mounting medium (VMRD, USA).
(5) Periodic acid-Schiff (PAS) staining
The kit Periodic-Acid-Schiff (Shanghai Yuanye Biotechnology Co., Ltd.) was used.
(6) UGT2B7 metabolism test
Reference substance: 1.0 mg/mL zidovudine solution (AZT. Sigma); 3′-azido 3′-deoxythymidine β-D-glucuronide (AZT-5′-glucuronic acid), Toronto Research Chemicals. Canada).

4. Primers

Primers used in the examples are shown in Table 2.

TABLE 2

| Gene | Forward (5′-3′) | Reverse (5′-3′) |
|---|---|---|
| ALB | gcctttgctc | aggtttgggt |
| | agtatctt | tgtcatct |
| | (SEQ ID NO: 1) | (SEQ ID NO: 2) |
| AAT | tatgatgaag | cagtaatgga |
| | cgtttaggc | cagtttgggt |
| | (SEQ ID NO: 3) | (SEQ ID NO: 4) |
| FAH | cctacggcgt | ctgcaagaac |
| | cttctcgac | actctcgcct |
| | (SEQ ID NO: 5) | (SEQ ID NO: 6) |
| G6PC | ctggagtcct | cagtcccttg |
| | gtcaggcatt | agcagcagat |
| | g | aa |
| | (SEQ ID NO: 7) | (SEQ ID NO: 8) |
| CYP2A6 | cagcacttcc | aggtgactgg |
| | tgaatgag | gaggacttga |
| | (SEQ ID NO: 9) | ggc |
| | | (SEQ ID NO: 10) |
| CYP2EI | ccccagcggc | tgggccaacc |
| | accatgtctg | gggtgaagga |
| | (SEQ ID NO: 11) | a |
| | | (SEQ ID NO: 12) |
| CYP3A4 | ttcagcaaga | ggttgaagaa |
| | agaacaagga | gtcctcctaa |
| | caa | gc |
| | (SEQ ID NO: 13) | (SEQ ID NO: 14) |
| CYP3A7 | tacacaccct | caaagcgtaa |
| | ttggaagtgg | tttcaggggg |
| | (SEQ ID NO: 15) | (SEQ ID NO: 16) |
| CYP7A1 | tgtccatttc | gtggtatttc |
| | atcacaaatc | catccatcgg |
| | ccttg | gtc |
| | (SEQ ID NO: 17) | (SEQ ID NO: 18) |
| SLCO1B1 | ttggaggtgt | acaagtggat |
| | tttgactgct | aaggtcgatg |
| | t | ttg |
| | (SEQ ID NO: 19) | (SEQ ID NO: 20) |
| UGT2B7 | tcagccctgg | acagctgctc |
| | cccagatccc | ccctggcctt |
| | (SEQ ID NO: 21) | (SEQ ID NO: 22) |
| AFP | actgaatcca | tgcagtcaat |
| | gaacactgca | gcatctttca |
| | (SEQ ID NO: 23) | (SEQ ID NO: 24) |
| ACTB | tggcacccag | ctaagtcata |
| | cacaatgaa | gtccgcctag |
| | (SEQ ID NO: 25) | aagca |
| | | (SEQ ID NO: 26) |
| Rat Afp | tgatttctct | ttctcctttc |
| | ggcctcttgg | ttcctcctgg |
| | (SEQ ID NO: 27) | (SEQ ID NO: 28) |
| Rat Actb | gagtacaacc | ccttctgacc |
| | ttcttgcagc | catacccacc |
| | tcc | at |
| | (SEQ ID NO: 29) | (SEQ ID NO: 30) |

5. Antibodies

Antibodies used in the examples are shown in Table 3.

6. HE Staining

TABLE 3

| | Commercial | Source | | Dilution |
|---|---|---|---|---|
| Antibody | source | species | Reactivity | ratio |
| Albumin | Bethyl | Goat | Human | 1:1000 |
| | Laboratories | | | |
| hNuclei | Millipore | Mouse | Human | 1:200 |
| AAT | Dako | Rabbit | Human | 1:1000 |
| FAH | Abbomax | Rabbit | Human, rat | 1:3000 |
| CK19 | Progen | Mouse | Human, rat | 1:100 |
| GS | BD | Mouse | Human, rat | 1:500 |
| CYP3A4 | Santa Cruz | Mouse | Human | 1:200 |
| CYP1A2 | Bio-Rad | Rabbit | Human | 1:200 |
| MRP2 | Genetex | Mouse | Human, rat | 1:200 |
| ARG1 | Genetex | Rabbit | Human, rat | 1:200 |
| UGT2B7 | Thermo Fisher | Rabbit | Human, rat | 1:200 |
| | Scientific | | | |
| Ki67 | Abcam | Rabbit | Human, rat | 1:200 |
| AFP | Dako | Rabbit | Human, rat, | 1:100 |
| | | | mouse | |
| Biotinylated goat | Vector | Goat | Rabbit | 1:200 |
| anti-Rabbit IgG | Laboratories | | | |
| Alexa Fluor 488- | Thermo Fisher | Goat | Mouce | 1:500 |
| conjugated goat | Scientific | | | |
| anti-mouse IgG1 | | | | |
| Alexa Fluor 647- | Thermo Fisher | Goat | Mouce | 1:500 |
| conjugated goat | Scientific | | | |
| anti-mouse IgG2b | | | | |
| Alexa Fluor 488- | Thermo Fisher | donkey | Rabbit | 1:500 |
| conjugated donkey | Scientific | | | |
| anti-rabbit IgG | | | | |
| Cy3-conjugated | Jackson Lab | donkey | Mouse | 1:500 |
| donkey anti-mouse | | | | |
| IgG | | | | |
| Alexa Fluor 647- | Thermo Fisher | donkey | Goat | 1:500 |
| conjugated donkey | Scientific | | | |
| anti-goat IgG | | | | |

Rat livers were fixed overnight in 4% PFA, dehydrated, embedded and cut into 3 um-thick sections.
(1) Xylene dewaxing for 3 times, 5 minutes each time.
(2) Gradient rehydration: 100% ethanol, 100% ethanol, 90% ethanol, 80% ethanol, 70% ethanol, 50% ethanol, each for 5 minutes. Deionized water, soak for 1 minute.
(3) Antigen retrieval (this step is optional according to the characteristics of the antibody): sodium citrate antigen retrieval buffer with pH=6.0 was heated at 95 degrees for half an hour, and then naturally cooled to room temperature. Wash with deionized water for 15 minutes.
(4) Hematoxylin counterstaining for 20 minutes. Rinse with tap water for half an hour.
(5) Gradient dehydration: 50% ethanol, 70% ethanol, 80% ethanol, 90% ethanol, 100% ethanol, each for 2 minutes.
(6) After 15-30 minutes of xylene, sections were sealed with neutral resin and saved.

7. Immunohistochemistry
(1) The steps of dewaxing and rehydration are the same as above.
(2) Draw a circle around the sample with an ImmunoPen, drop a drop of 3% H₂O₂ for 15 minutes at room temperature for blocking endogenous peroxidase.
(3) Wash twice with PBS for 5 minutes each time.
(4) The blocking was carried with 1:20 Normal horse serum (dissolved in 1% BSA-PBS), incubated at room temperature for 20 minutes and washed for 3 times with PBS.
(5) The primary antibody was added, then the slices were put in a humid box, sealed and kept overnight at 4 degrees.
(6) Wash three times with PBS for 5 minutes each time.
(7) Secondary antibody was added and incubated at room temperature for 30 minutes. Wash three times with PBS for 5 minutes each time.
(8) AB solution in ABC kit (1:100 dilution of solution A and B) was added and incubated at room temperature for 30 minutes. Wash three times with PBS for 5 minutes each time.
(9) DAB coloration was carried and the time for coloration was adjusted according to different antibodies.
(10) Hematoxylin counterstaining for 20 minutes. Rinse with tap water for half an hour.
(11) Gradient dehydration: 50% ethanol, 70% ethanol, 80% ethanol, 90% ethanol, 100% ethanol, each for 2 minutes.
(12) After 15-30 minutes of xylene, sections were sealed with neutral resin and saved.

8. Immunofluorescence Staining
(1) Frozen sections (7 μm) were fixed in 4% PFA at room temperature for 10 minutes, or in methanol:acetone (1:1) at −30° C. for 30 minutes.
(2) Wash three times with 0.1% tween-PBS for 5 minutes each time.
(3) 10% goat or donkey serum was used for blocking for 60 minutes at room temperature.
(4) The primary antibody was used for incubation overnight at 4° C.
(5) Wash three times with 0.1% tween-PBS for 5 minutes each time.
(6) The secondary antibody conjugated with fluorescein was used for incubation at room temperature for 60 minutes.
(7) Wash three times with PBS for 5 minutes each time.
(8) DAPI was added and the sections were mounted by mounting medium and preserved.

9. Quantitative qPCR

1 μg RNA was reverse-transcribed into cDNA with Revert Aid RT kit according to manufacturer's instructions. According to the instructions of THUNDERBIRD SYBR qPCR Mix kit, cDNA was amplified and quantitatively analyzed on a quantitative PCR machine. Relative expression levels of target genes were based on ACTB as an internal reference. All data were repeated at least twice.

10. Liver Function Test

The content of alanine aminotransferase (ALT) and aspartate aminotransferase (AST) in rat serum was measured according to the instructions of biochemical analyzer FUJI DRI-CHEM 7000V (Fujifilm. Japan).

11. Measurement of Human Albumin Secretion

100 μl of rat peripheral blood was collected through the tail vein, and after coagulation at room temperature, centrifuged at 400-g for 10 minutes, with the supernatant collected to obtain serum. The secretion level of human albumin was tested according to the instructions of the Human Albumin ELISA Quantitation Set kit. Serum was diluted 10-100000 times according to the actual concentration, so that the calculated value falls within the linear range of the standard curve to obtain accurate values.

12. Primary Hepatocyte Transplantation and Programmed NTBC Circulation
(1) Preparation before transplantation: Female FRG rats aged 7-8 weeks were intraperitoneally injected with 30 mg/kg Retrorsine 2 weeks before transplantation, and the concentration of NTBC in daily water was reduced from the normal maintenance concentration (5˜8 mg/L) to low concentration (0.2 mg/L) 1 week before transplantation. When the programmed NTBC cycle began, daily NTBC water was provided according to the method of 0.2 mg/L×7 days+5 mg/L×4 days (one cycle period of 11 days in total).

In the past feeding, FRG rats were daily supplied with 5-8 mg/L. In the present disclosure, the lowest dose of 5 mg/L NTBC was set as 100% NTBC; According to the weight (250-300 g) and the amount of water (25-50 ml/day) of rats, we can get:
4% NTBC: equivalent to an average of 0.03 mg/kg/day;
100% NTBC: equivalent to an average of 0.75 mg/kg/day.
(2) The transplantation was performed under an anesthesia machine using isoflurane. The rat was fixed with its abdomen facing upward, and cleaned and disinfected with iodine and alcohol after dehairing.
(3) By cutting the epidermis and muscle layers, the hepatic portal vein was found. 2×10⁶ human primary hepatocytes (resuspended in 800 μl DMEM+10% FBS) were slowly injected with a 26G needle. After completing the injection and paused for a few seconds, the needle was then pulled out, with the needle port quickly pressed with a sterile cotton swab.
(4) After pressing for about 1 minute, with no blood effusion be observed, the muscle and epidermis were sutured and the wound was disinfected with alcohol cotton.

13. UGT2B7 Metabolism Test In Vivo

Liver humanized and uncontrolled FRG rats were orally administered 15 mg/kg zidovudine (Wako), and peripheral blood was collected after 0, 0.5, 1, 2, 4 and 8 hours. After 30 μl of plasma was diluted 2 fold with PBS, the contents of substrates and metabolites were detected by LC-MS/MS (Agilent 1200 HPLC and ABI 4000 mass-spectrometer).

### Example 1. FRG Rats Develop Fatal Liver Injury after NTBC Withdrawal

After long-term follow-up observation, the inventors found that FRG rats could survive healthy for at least 1.5 years under the maintenance of 100% NTBC (Daily supply is 5-8 mg/L for FRG rats. In the present disclosure, the minimum dose of 5 mg/L is set as 100%), providing a sufficient time window for the construction of humanized livers (FIGS. 1A and D). After NTBC withdrawal, rats developed acute liver injury, evidenced by elevated levels of ALT and AST (FIG. 1B) as well as necrosis and fibrosis in their livers (FIG. 1C). All rats died within 4 weeks after NTBC withdrawal and the median survival time was 9.6 days (FIG. 1D).

The results showed that NTBC withdrawal can cause severe liver injury in FRG rats, and then lead to death.

This observation in rats is different from that in other animals, such as mice, which maintain a longer period of survival after NTBC withdrawal and maintain a higher level of liver injury. Therefore, for rats, some existing NTBC application protocols in the art are not applicable.

### Example 2. Establishment of NTBC Programmed Regulation to Induce Chronic Liver Injury

In order to improve the survival rate of FRG rats under the condition of liver injury and ensure the stability of the long-term humanization process, the inventors conducted in-depth research and analyzed the liver injury and its survival in rats from multiple perspectives, in order to explore and improve the optimal conditions for survival in rat liver injury.

The optimal NTBC maintenance concentration was determined by comparing the survival rate of rats at different NTBC concentrations (0%, 1%, 4%, 10%, 100%). The results showed that when the NTBC concentration exceeded 4% of the maintenance dose (5 mg/L), the median survival time was more than three weeks (22 days), which could effectively prevent sudden death in rats (FIG. 2A). Further comparison of liver function and body weight status of rats under 4% and 10% NTBC conditions showed that 4% conditions could maintain higher levels of ALT and AST, and significantly inhibit the increase of body weight in rats (FIG. 2B), achieving both maintenance of survival and inducement of liver injury.

Due to a long period of the humanization process and in order to further improve the long-term survival rate of rats under liver injury, the inventors designed a series of NTBC dosage regimens, which are called Programmed NTBC controlling scheme with a fixed cycle period (Programmed NTBC controlling, NTBC^{P}), including 5, 7 and 9 days of maintenance in 4% NTBC conditions, respectively, and 4 days of 100% NTBC recovery after that to alleviate accumulated liver injury (FIG. 2C).

The results showed that FRG rats were all maintained a high survival rate within 2 months under the controlling of programmed NTBC 5+4 (5 days of 4% NTBC+4 days of 100% NTBC cycle) and 7+4 (7 days of 4% NTBC+4 days of 100% NTBC cycle) (FIG. 2D).

Further comparing the two modes of 5+4 and 7+4, the inventors found that 7+4 had significantly higher levels of ALT and AST (FIG. 2E), which could better induce chronic liver injury; By observing the degree of liver fibrosis (FIG. 2F), the degree of fibrosis was more significant under 7+4.

At the same time, the inventors also observed that the long-term effect of programmed NTBC controlling did not lead to abnormal upregulation of the rat liver tissue-specific oncogene Afp (FIG. 2G), proving that this strategy is safe.

Based on the above results, the inventors established a controlling strategy of 7+4 NTBC^{P} that can continuously induce chronic liver injury, and applied it to the subsequent production of rat models of humanized livers.

### Example 3. Retrorsine Pretreatment Combined with Programmed NTBC Controlling Scheme

The inventors found that in the process of programmed NTBC controlling scheme (7+4: 7 days of 4% NTBC+4 days of 100% NTBC cycle), after transplantation of primary human hepatocytes (PHH), levels of repopulated and amplified PHH in the humanized rat model are still suboptimal. In order to achieve further optimization, the inventors conducted extensive analysis and experiments on a wide variety of target drugs, and found that pretreatment with Retrorsine (RS) has a facilitating effect.

The inventors set up two groups of hepatocytes in FRG rats with (w/RS) and without (w/o RS) Retrorsine (RS) pretreatment to observe the levels of repopulated and amplified PHH in the rat model.

After 2 months of programmed NTBC controlling as described in Example 2, it was found that the proportion of Ki67 positive cells in rat livers with RS-pretreatment was significantly lower (FIGS. 3A and B). The secretion level of human albumin (hAlbumin. ALB) is an important indicator to evaluate the level of repopulated and amplified humanized hepatocytes in vivo. By comparison, it was found that the albumin secretion level increased significantly in rats with RS pretreatment after transplanted with primary human hepatocytes (PHH) (FIG. 3C), indicating that the combination of RS pretreatment and programmed NTBC controlling scheme can better promote PHH repopulation and expansion in vivo.

### Example 4. Achievement of Humanized Rat Liver Under Chronic Liver Injury

After human primary hepatocyte transplantation in rats as described above, survival curves of FRG rats after transplantation of PHH in previous programmed NTBC controlling scheme with a fixed cycle period called NTBC^{P}(7+4: 7 days of 4% NTBC+4 days of 100% NTBC cycle; and Retrorsine pretreatment) and in NTBC^{ΔBW} with the change of 10% bodyweight as an index (withdrawing NTBC, when the body weight in rats decreased by 10%. 100% NTBC was given, and when the bodyweight recovered to the bodyweight before withdrawal. NTBC was withdrawn, and the cycle was repeated) were compared (FIG. 4A).

The results showed that rats after transplantation had a higher survival rate under NTBC^{P} controlling scheme, with 6/7 of the rats surviving for at least 20 weeks (FIG. 4B). Under the NTBC^{P} controlling, the secretion level of human albumin in surviving rats continued to increase over time and reached a maximum of 2.2 mg/ml (mean 1.7±0.3 mg/ml) after 7 months (FIG. 4C).

By immunostaining of the human-specific marker hNuclei, the repopulation rates of humanized rat livers can be calculated. As shown in FIG. 4D, under NTBC^{P} controlling scheme, the repopulation rate of human cells after transplantation continued to increase, and the repopulation rate reached 31±4% at 7 months (FIG. 4E).

### Example 5. Cellular Identification of Humanized Liver

Under the programmed NTBC controlling scheme, NTBC^{P}, (7+4: 7 days of 4% NTBC+4 days of 100% NTBC cycle; and Retrorsine pretreatment) mentioned above, repopulated human cells were obtained and identified 7 months after transplantation of PHH in FRG rats. The inventors used hALB, hAAT and hNuclei antibodies to counterstain and observe the antigens specific to human hepatocytes. The results showed that all hNuclei-positive human cells expressed hALB and hAAT, indicating that these cells were mature hepatocytes (FIG. 5A).

In addition, FAH positive human hepatocytes showed whiter color than that of surrounding area in rat livers by HE staining (FIG. 5B), which can clearly distinguish the sources of two different species. Meanwhile, human hepatocytes also exhibited increased accumulation of glycogen (FIG. 5C). Histologically, it was confirmed that the repopulation of human hepatocytes did not affect the original liver structure of the rat (FIG. 5B). These results are consistent with related reports in humanized mouse models. At the same time, the results of counterstaining with CK19 and hNuclei antibodies showed that there were no CK19-positive human cells, indicating that the transplanted human primary hepatocytes were not transformed into biliary epithelial cells in rats (FIG. 5D). In addition, by analyzing the proportion of Ki67-positive cells in hALB-positive hepatocytes, it was found that the proliferation of human hepatocytes gradually decreased over time (Figure SE). However, the ratio of more than 10% was still maintained after 7 months of transplantation (Figure SF), indicating that NTBC^{P} can induce human primary hepatocytes to acquire long-term expansion potential in vivo.

### Example 6. Detection of Humanized Liver Tumorigenesis

Under the programmed NTBC controlling scheme, NTBC^{P}, (7+4: 7 days of 4% NTBC+4 days of 100% NTBC cycle; and Retrorsine pretreatment) mentioned above, humanized livers were obtained and liver/bodyweight ratios were calculated 7 months after transplantation of PHH in FRG rats.

The results showed that the liver/bodyweight ratios were not significantly increased when compared with nontransplanted controls (FIG. 6A). Histology revealed no tumor formation. By further analyzing the gene and protein levels of a marker of hepatic carcinoma, AFP, the results showed that there was no abnormal up-regulation of AFP after repopulation of human hepatocytes into livers of rats for 7 months (FIGS. 6B and C).

The above results indicated that under the condition of NTBC^{P}, malignant lesions were not induced in the process of liver humanization.

### Example 7. Humanized Liver Shows Metabolism-Related Gene and Protein Expression Similar to Human Liver

Under the programmed NTBC controlling scheme, NTBC^{P}, (7+4: 7 days of 4% NTBC+4 days of 100% NTBC cycle; and Retrorsine pretreatment) mentioned above, functions of humanized livers in rats were evaluated 7 months after transplantation of PHH in FRG rats.

The present inventors first performed QPCR analysis of the expression of metabolism-related genes using primers (Table 2) that specifically recognize human genes. The results showed that the humanized liver not only expressed mature hepatocyte marker-related genes (ALB, AAT, G6PC. Fah), but also expressed phase I metabolic enzymes (CYP2A6, CYP2E1, CYP3A4, CYP3A7 and CYP7A1), phase II metabolic enzymes (UGT2B7) and transporter related genes (SLC22A4 and SLCO1B1) (FIG. 7A). More importantly, its expression level was close to that of PHH before transplantation.

Orderly distribution of metabolic enzymes in livers is an important structural feature of liver. By immunostaining of randomly selected liver metabolic enzymes, it revealed that glutamine synthase (GS) was specifically distributed on hepatocytes near the central vein (FIG. 7B), the phase I metabolic enzyme cytochrome P450 3A4 enzyme (CYP3A4). 1A2 enzyme (CYPIA2) are also concentrated around the central vein. In contrast, arginase (ARG1) was mainly concentrated near the hepatic portal vein (FIG. 7B). In addition, the phase II enzymes UDP-glucuronosyl transferase 2B79 (UGT2B7) and the transporter multidrug resistance-associated protein 2 (MRP2) were evenly distributed throughout the liver lobules (FIG. 7C). Importantly, the distribution of above metabolic enzymes in humanized livers is consistent with the distribution in vivo.

The above results showed that the humanized liver showed great similarity with the human liver in terms of the expression characteristics of metabolism-related genes and enzymes.

### Example 8. Humanized Liver has Human-Specific Drug Metabolism Characteristics

It is known that the human UGT2B7 enzyme is involved in the metabolism of about 35% of clinical drugs, which has important research significance. The inventors used UGT2B7 metabolized drug zidovudine (AZT) to verify whether rats with liver humanization can exhibit human-specific metabolic characteristics. In human, 75% of AZT can be metabolized to AZT-5′-glucuronide, while in rats, the metabolic conversion rate is only 10%. FIG. 8A shows the testing process.

The results showed that after oral administration of AZT. AZT-5′-glucuronide in rats with liver humanization (about 30% repopulation rate) was significantly higher than that in the control group (FIG. 8B). The AUC (areas under the curves) of AZT-5′-glucuronide/AZT were 39%±16% and 6%±3% in rats with liver humanization and rats in the control group, respectively (FIG. 8C). In addition, the comparison of the correlation between the amount of human albumin secretion and the AUC (AZT-5′-glucuronide/AZT) also showed that the liver humanization rate was positively correlated with the human-type metabolic level of AZT (FIG. 8D).

The above results indicate that the rats with liver humanization exhibit unique characteristics of human drug metabolism in vivo, with the ability to survive for a long time.

### Example 9. Kit/Set for the Preparation of Humanized Livers in Animals

In this example, a kit for preparing humanized livers in rats is provided, wherein it comprises:
Container group 1, comprising a container and a low dose of Nitisinone in the container, wherein the low dose is 0.03 mg; the container group 1 has 7 containers;
Container group 2, comprising a container and a high dose of Nitisinone in the container, wherein the high dose is 0.75 mg; the container group 2 has 4 containers;
Container group 3, comprising a container and Retrorsine in the container, wherein the dose is 4 mg; the number of containers in the container group 3 is 1;
The kit was used for a single (11-day) dosing and pretreatment of rats with Retrorsine.

After a plurality of the kits are integrated, they can be used for multiple cycles of rat administration, comprising simultaneous administration of multiple rats. In continuous animal preparation, a set of kits can be used, for example, the set of kits contain 2 to 200 kits. In the set of kit composed of multiple kits. Retrorsine only needs to be provided in one kit.

Each reference provided herein is incorporated by reference to the same extent as if each reference was individually incorporated by reference. In addition, it should be understood that based on the above teaching content of the disclosure, those skilled in the art can practice various changes or modifications to the disclosure, and these equivalent forms also fall within the scope of the appended claims.

1. A method for maintaining chronic liver injury in a Fah gene-deficient animal model, wherein the method comprises administering Nitisinone to the Fah gene-deficient animal model according to the following scheme:
(1) administering a low dose of Nitisinone every day for 3-12 days, wherein the low dose is 0.005-0.1 mg/kg animal body weight/day;
(2) administering a high dose of Nitisinone every day for 2-6 days, wherein the high dose is 0.25-1.5 mg/kg animal body weight/day;
(3) repeat the cycle of step (1) and (2).

2. A method for preparing a heterologous liver transplantation animal model, the animal model has a heterologous liver, the method comprising:
(1) administering a low dose of Nitisinone every day for 3-12 days, wherein the low dose is 0.005-0.1 mg/kg animal body weight/day; (2) administering a high dose of Nitisinone every day for 2-6 days, wherein the high dose is 0.25-1.5 mg/kg animal body weight/day;
(3) repeat the cycle of step (1) and (2);
and on the 3rd to 30th days after the initiation of the above scheme, the animals were transplanted with heterologous hepatocytes.

3. The method according to claim 1 or 2, wherein, before carrying out the scheme, it further comprises:
pretreating the animal with Retrorsine; preferably, the dose of Retrorsine is 10-50 mg/kg animal body weight; more preferably, the dose of Retrorsine is 2040 mg/kg animal body weight; further preferably, the dose of Retrorsine is 25-35 mg/kg animal body weight.

4. The method according to claim 1 or 2, wherein, in step (1), administering a low dose of Nitisinone every day for 4-10 days; preferably, administering a low dose of Nitisinone every day for 5, 6, 7, 8 or 9 days; or
in step (1), the low dose is 0.008-0.08 mg/kg animal body weight/day; preferably, the low dose is 0.01-0.05 mg/kg animal body weight/day.

5. The method according to claim 1 or 2, wherein, in step (2), administering a high dose of Nitisinone every day for 3-5 days; or
in step (2), the high dose is 0.3-1.2 mg/kg animal body weight/day; preferably, the high dose is 0.35-1 mg/kg animal body weight/day.

6. The method according to claim 1 or 2, wherein the animal is a mammal that is at least twice larger in size than a mouse; preferably, the animal comprises an animal selected from the group consisting of: rat, rabbit, monkey, pig, guinea pig, dog; preferably, the animal is an animal wherein the interleukin 2 receptor gamma gene and the recombination activating gene 2 are destroyed.

7. The method according to claim 2, wherein, the heterologous hepatocytes comprise: hepatocytes that belongs to different species from the animal model and derived from human, mouse, pig, monkey or dog: preferably, the heterologous hepatocytes comprise:
(a) primary hepatocytes;
(b) hepatic stem/progenitor cells, hepatocytes or hepatocyte-like cells, endoderm cells, etc. induced and differentiated from induced pluripotent stem cells and embryonic stem cells;
(c) hepatic stem/progenitor cells, hepatocytes or hepatocyte-like cells induced and differentiated from stem cells of endoderm and other germ layers;
(d) hepatic stem/progenitor cells, hepatocytes or hepatocyte-like cells derived from direct transdifferentiation of adult or fetal stem/progenitor cells or hepatocytes and other somatic cells;
(f) hepatic stem/progenitor cells, hepatocytes or hepatocyte-like cells after in vitro induced expansion of cells derived from (a)-(d);
(g) liver organoids constructed from cells derived from (a)-(f).

8. A system for preparing a Fah gene-deficient animal model and maintaining chronic liver injury, comprising:
an administration component or module 1, wherein it is set to administer a low dose of Nitisinone to animals every day for 3-12 days, wherein the low dose is 0.005-0.1 mg/kg animal body weight/day;
an administration component or module 2, wherein it is set to administer a high dose of Nitisinone to animals every day for 2-6 days, wherein the high dose is 0.25-1.5 mg/kg animal body weight/day.

9. The device according to claim 8, wherein it is also used for preparing a heterologous liver animal model with a heterologous liver, the device further comprises:
an administration component or module 3, wherein it can administer Retrorsine to animals; preferably, it is set to administer Retrorsine at 10-50 mg/kg animal body weight.

10. A kit for preparing a Fah gene-deficient animal model and maintaining chronic liver injury, wherein it comprises:
container group 1, comprising a container and a low dose of Nitisinone in the container, wherein the low dose is 0,005-0.1 mg/kg animal body weight; preferably, the number of containers in the container group 1 is 3-12;
container group 2, comprising a container and a high dose of Nitisinone in the container, wherein the high dose is 0.25-1.5 mg/kg animal body weight; preferably, the number of containers in the container group 2 is 2-6;
preferably, if a single kit is used for 1 cycle of administration, in continuous animal preparation, a set of kits can be used, for example, the set of kits contain 2-200 kits.

11. The kit according to claim 10, wherein, the kit also comprises:
container 3, comprising a Retrorsine in the container, preferably, the dose of Retrorsine is 1.2-10 mg.

12. A use of low-dose Nitisinone, high-dose Nitisinone and Retrorsine, for:
maintaining chronic liver injury in a Fah gene-deficient animal model; or preparing an administration group and a set of kits for maintaining chronic liver injury in a Fah gene-deficient animal model; and/or
preparing a heterologous liver animal model, wherein the model has a heterologous liver; or preparing an administration group and a set of kits for the preparation of a heterologous liver animal model;
wherein, the low dose is 0.005-0.1 mg/kg animal body weight/day; the high dose is 0.25-1.5 mg/kg animal body weight/day.
